(19) United States
(12) Patent Application Publication
(10) Pub. No.: US 2019/0167714 A1
(43) Pub. Date: Jun. 6, 2019

(54) HYDROGEL COMPOSITIONS AND USES THEREOF

(71) Applicant: Zoetis Services LLC, Parsippany, NJ (US)

(72) Inventors: Christina Lee Brown, Kalamazoo, MI (US); ALEXANDRE THERRIEN, Montreal (CA); NICOLAS COTTENYE, Montreal (CA); OSCAR SUAREZ, Montreal (CA)

(21) Appl. No.: 16/203,872

(22) Filed: Nov. 29, 2018

Related U.S. Application Data
(60) Provisional application No. 62/593,407, filed on Dec. 1, 2017.

(51) Int. Cl. A61K 31/722; A61K 47/12; A61K 47/02; A61K 47/10; A61K 9/06

(52) U.S. Cl. CPC A61K 31/722; A61K 47/12; A61P 15/14; A61K 47/10; A61K 9/06; A61K 47/02

(57) ABSTRACT

The present application provides compositions configured to be provided in a flowable, liquid form but that are configured to transition to a solid or semi-solid gel based upon a change in pH. The change in pH can arise from one or more materials utilized in the composition itself or based upon encountering an environment with a significantly different pH. The compositions are particularly suited for use as a teat sealant in non-human mammals, particularly cattle.

### CROSS REFERENCE TO RELATED APPLICATION

This application claims the benefit of U.S. Provisional Patent Application No. 62/593,407, filed Dec. 1, 2017, which is expressly incorporated herein by reference in its entirety.

### FIELD OF THE DISCLOSURE

The present disclosure relates to compositions that are configured to exhibit pH-dependent properties, as well as methods of using such compositions. More particularly, the compositions can be configured to form a solid or semi-solid gel at about physiological pH and be a flowable liquid at lower pH.

### BACKGROUND

Hydrogels are highly hydrated, macromolecular networks, dispersed in water or other biological fluids. The complexity of biological structures such as natural tissues has resulted in researchers exploring the use of biomaterials and medical devices that are introduced on the skin or into the body of a subject as a liquid and that turn solid or solid-like during or after application or injection. For example, chitosan hydrogels have been shown to be useful for cartilage regeneration and prevention of knee pain associated with acute and chronic cartilage defects. Chitosan-based gels have also been shown to turn into and serve as scaffolds for the encapsulation of invertebral disc (IVD) cells by entrapping large quantities of newly synthesized anionic proteoglycan. Chitosan is known to form thermoreversible gels in the presence of several multivalent anions, such as phosphate derivatives. Temperature-controlled pH-dependent formation of ionic polysaccharide gels, such as chitosan/organo-phosphate aqueous systems, has been described, for example, in PCT International Publication No. WO 99/07416 and U.S. Pat. No. 6,344,488. However, hydrogels made from ionic polysaccharides such as chitosan are weak and usually form only after a relatively long waiting time, after mixing polymer and salt solutions. This is mainly due to the fact that it is difficult to obtain homogenous, fully-hydrated chitosan solutions with a high concentration of chitosan, especially high molecular weight chitosan, due to its poor solubility. Furthermore, several medical applications require a solid structure with desired macroporosity and mechanical properties. Thus, there is a need for stimuli-responsive implants and patches that can reach desired mechanical and/or permeability properties only when triggered by specific physiological stimuli.

U.S. Pat. No. 9,034,348 discloses injectable chitosan mixtures forming hydrogels. There are described chitosan compositions which form a hydrogel at near physiological pH and 37° C. comprising at least one type of chitosan having a degree of acetylation in the range of from about 30% to about 60%, and at least one type of chitosan having a degree of deacetylation of at least about 70%. Further disclosed is a chitosan composition which forms a hydrogel at near physiological pH and 37° C. that includes at least one type of chitosan having a degree of deacetylation of at least about 70% and a molecular weight of from 10-4000 kDa. and at least one type of a chitosan having a molecular weight of from 200-20000 Da. Also disclosed are methods of preparation and uses of the chitosan compositions.

U.S. Patent Application Publication No. 2010/0028434 discloses temperature-controlled and pH-dependent self-gelling biopolymeric aqueous solutions. There are described biopolymeric liquid aqueous compositions for producing self-gelling systems and gels, which comprises an acidic water-based medium, 0.1 to 10% by weight of a pH-gelling acid-soluble biopolymer, and 0.1 to 10% by weight of a water-soluble molecule having a basic character and a pKa between 6.0 and 8.4, or a water-soluble residue or sequence of the molecule having a basic character and a pKa between 6.0 and 8.4. The liquid compositions have a final pH ranging from 5.8 to 7.4, and form a stable solid and homogeneous gel within a temperature range from 10 to 70° C. Methods for preparing the compositions and uses thereof are also described.

U.S. Patent Application Publication No. 2010/0285113 discloses inverse thermal gelling composite hydrogels having enhanced stability. There are described composite hydrogels comprising a blend of an aqueous solution of an anionic polysaccharide or a derivative thereof, such as hyaluronan (also commonly referred to as hyaluronic acid) or a derivative thereof and an aqueous solution of methylcellulose or another water soluble cellulose derivative thereof, having dispersed polymeric particles, such as polymeric hydrophobic particles therein selected from microparticles and nanoparticles, and wherein the stability of the hydrogel is enhanced relative to the stability of the hydrogel alone. The polymeric particles may contain at least one therapeutic agent, in which case each therapeutic agent exhibits a linear sustained release rate that can be tuned or altered by selecting the appropriate polymer formulation of the microparticles and/or nanoparticles. The composite may be injectable, and in the absence of a therapeutic agent may be used as a bulking agent for reconstructive and cosmetic surgery or may act as a platform for subsequent delivery of therapeutic agents.

Insofar as veterinary health issues are concerned, mastitis is an inflammation of the mammary gland that is typically caused by bacteria which in most cases enter the gland via the teat orifice. During the non-lactating period or “dry period” in the gland, deposits of keratin in the teat orifice and the streak canal form a primary defense mechanism. A keratin plug that forms in the teat of the animal forms a protective barrier, and the immune-rich tissues of the Furstenburg's Rosette in the teat, as well as the natural protective factors of the dry-cow secretions, contain high levels of naturally occurring anti-bacterial substances (cationic proteins) which inhibit the passage of bacteria from the teat orifice to the teat cistern (papillary sinus) and gland cistern. However, this keratin plug and these natural immune defense mechanisms can be overcome by bacterial invasion as the animal enters into the dry period at the end of lactation, during the dry period of the animal, and/or during calving. As a result, bacteria invade the gland and cause mastitis during the dry period or, more particularly, immediately following calving.

The major pathogens causing mastitis are Staphylococcal species such as, for example, Streptococcus agalactiae, Staphylococcus aureus and the like, Coryne bacterium bovis, Mycoplasma, coliforms such as, for example, Esherichia coli, Klebsiella spp., Enterobacter spp., and Citrobacter spp., environmental Streptococcal species such as, for example, Strep. dysgalactiae. Strep, uberis, and Enterococcus spp., Pseudomonas spp., etc. Although mastitis is mainly caused by bacteria, the inflammation can also be produced as a result of viral infection (e.g., bovine herpesvirus II and IV, a paravaccinia virus such as Pseudo Cowpox. and the like) or infection with atypical pathogens like mycotic (e.g., Candida spp. and Aspergillus spp.) or algal microbes (e.g., Prototheca spp.) with or without development of a secondary bacterial infection.

Mastitis due to the presence of pathogens can become a highly contagious condition within the confines of a dairy farm that results in huge production losses for the dairy industry. Reduction of drinkable milk then occurs from the harmful pathogens' effects or various treatments that render the milk not fit for human consumption. While severe cases can end in death, unhindered outbreaks can also cause permanent damage to the animals' udders. As a major endemic disease of dairy animals, mastitis puts the animal welfare at risk and often entails rather costly veterinary care. The value of protecting the early lactation period from existing and new infections perpetuated from the dry period remains highly valuable to the industry. It is clear that the treatment and control of mastitis is an important goal to maintain the animal's health and to lower the high costs of milk production in the dairy industry.

To that end, products have been developed in an attempt to seal an animal's teat to prevent mastitis and other conditions, for example, barrier teat dips to seal the external surface and streak canal of the teat during periods of milking and internal teat sealants to block or to seal the teat canal or to plug the teat cistern during the dry period, especially for heifers and cows that have experienced one or more pregnancies previously.

Along with these products, several methods to reduce the incidence of mastitis are described in the art, for example, a method comprising sequentially delivering from a single delivery device an antimicrobial formulation and a seal formulation into the teat canal of a non-human animal wherein the seal formulation is nontoxic heavy metal salt such as bismuth (U.S. Pat. No. 8,353,877); a method of applying to the teat canal and/or teat sinus a composition comprising exogenous keratin (U.S. Pat. No. 8,226,969); a method of forming a physical barrier in the teat canal for prophylaxis during an animal's dry period by infusing an amount of a teat seal formulation into the teat canal of the animal, wherein the teat seal formulation comprises a bismuth-free, nontoxic, heavy metal salt of titanium, zinc, barium or combinations thereof and the physical barrier does not cause a black spot defect in dairy products made with milk from the animal (U.S. Pat. No. 7,906,138); a method of forming an anti-infective free physical barrier in the animal's teat canal for prophylactic treatment of mastitis during the dry period comprising the step of infusing a seal formulation into the teat canal of the animal without an anti-infective agent, wherein the seal formulation comprises a nontoxic heavy metal salt such as bismuth in a gel base of aluminum stearate with a vehicle such as liquid paraffin or a gel base comprising a polyethylene gel (U.S. Pat. No. 6,254,881) and the like.

None of the existing seal formulations or external dip products seals the teat of the dairy animal externally for a sufficient amount of time to prevent mastitis, particularly the form that can be fatal and/or very contagious in the animals, like among heifers. Moreover, while teat sealants have been established as a viable method to provide a higher level of protection regardless of antibiotic choice or administration, the current products on the market fail to meet the demand for ease of use and long-lasting tissue adherence, ease of removal, avoidance of milk contamination, and prevention of black spot defect in aged cheese. What is needed, therefore, is a nontoxic formulation that is easy and safe for the animal handler to administer and that preferably forms an effective, long-lasting seal in place directly on the tissue (that is, “in situ”). Additionally, it is necessary for the seal formulation not to interfere with the quality of the dairy animal's milk, yogurt or cheese products created from the milk, especially for the sealant to avoid the black spot defect in aged cheese. Indeed, there is a definite art-recognized need in the veterinary field to find a long-lasting, nontoxic, non-irritating seal formulation that forms an adequate barrier on the animal's teat to prevent or to reduce significantly the incidence of mastitis caused by pathogens, preferably without the use of antibiotics or other medicinal agents that require a withholding period for public consumption of the animal's milk. There is also a definite need to find a long-lasting seal formulation that can contain antibiotics and the like for the effective treatment or prevention of mastitis.

### SUMMARY OF THE DISCLOSURE

The present disclosure provides compositions that are configured for forming a durable seal and/or strong solid and that are thus suitable for use in a variety of medical and veterinary fields. Specifically, the compositions are suitable for forming a physical barrier in the teat canal of a dairy animal for the prophylactic treatment or prevention of mammary disorders that occur mainly as the animal enters the dry period or during the dry period. Beneficially, the composition can be provided in the form of a flowable liquid that can be externally applied to the teat of the animal and/or can be infused within the teat canal or cistern. Preferably, the composition gels or solidifies rapidly after application to form a strong solid. The composition can be combined with an application device and/or infusion device to form a system for application of the composition to a non-human mammal. The compositions are effective as a teat sealant to block the invasion of the mammary gland by a mastitis-causing microorganism or to decrease the occurrence or re-occurrence of infection.

In one or more embodiments, the present disclosure thus relates to a composition comprising: chitosan; a biocompatible acid; a filler; a fluidizing agent; and at least 50% by weight of an aqueous medium based upon the total weight of the composition; wherein the composition is a flowable liquid at a pH of less than 6 less and is a solid or semi-solid gel at a pH of about 6.5 or greater. In one or more embodiments, the composition may be further defined in relation to one or more of the following statements, which may be combined in any order or number.

The chitosan can be present in an amount of about 2% to about 5% by weight based on the total weight of the composition.

The chitosan can have a degree of deacetylation of greater than 90%.

The biocompatible acid can be present in an amount of about 0.5% to about 5% by weight based on the total weight of the composition.

The biocompatible acid can be an organic acid.

The biocompatible acid can be acetic acid.

The chitosan and the biocompatible acid can be present in a chitosan to acid weight ratio of about 1:1 to about 4:1.

The filler can be present in an amount of about 10% to about 40% by weight based on the total weight of the composition.

The filler can be selected from the group consisting of calcium phosphate, aluminum oxide, calcium carbonate, silicon dioxide, and combinations thereof.

The fluidizing agent can be selected from the group consisting of polyethylene glycol, glycerol, and combinations thereof.

The composition can be substantially free of any cellulosic material.

In further embodiments, the present disclosure can provide a method of treating a non-human mammal. For example, the method can comprise administering a composition as described herein to a teat, teat canal, or teat cistern of the non-human mammal.

### BRIEF DESCRIPTION OF THE DRAWINGS

The present disclosure can be further described with reference to the accompanying drawings, wherein:

FIG. 1 is a graph showing the correlation between dibasic phosphate salt content and solution pH in compositions according to embodiments of the present disclosure;

FIG. 2 is a graph showing the correlation between storage modulus (G′) and pH for compositions according to embodiments of the present disclosure;

FIG. 3 is a graph showing the relationship between storage modulus and pH at 25° C. for compositions according to embodiments of the present disclosure;

FIG. 4 is a graph showing erosion and dissolution of gels formed with compositions according to embodiments of the present disclosure after storage in cow milk at 37° C.; and

FIG. 5 is a graph showing the syringeability of a composition according to an embodiment of the present disclosure pre- and post-autoclave.

### DETAILED DESCRIPTION OF THE DISCLOSURE

Some aspects of the present disclosure will now be described more fully hereinafter with reference to the accompanying drawings, in which some, but not all implementations of the disclosure are shown. Indeed, various implementations of the disclosure may be expressed in many different forms and should not be construed as limited to the implementations set forth herein; rather, these exemplary implementations are provided so that this disclosure will be thorough and complete, and will fully convey the scope of the disclosure to those skilled in the art. As used in the specification, and in the appended claims, the singular forms “a”, “an”, “the”, include plural referents unless the context clearly dictates otherwise.

The present disclosure relates to compositions that are particularly suitable for use in mammary tissue, such as in non-human mammals. The compositions preferably comprise chitosan, a biocompatible acid, a multivalent salt, and an aqueous medium. The compositions are configured to transition between being a flowable liquid and being a substantially non-flowable solid or semi-solid gel based upon the pH of the composition and/or the pH of the environment of the composition. In one or more embodiments, the present composition can be configured to gel quickly upon pH change so as to provide a solid or semi-solid gel that is sufficiently strong to withstand mechanical or hydraulic pressures in physiological conditions. Additionally, the composition, when under conditions so as to be in the form of a solid or semi-solid gel, is configured for resisting and/or completely avoiding leaking and/or dripping. As such, after injection into a mammalian subject, the composition can be substantially in the form of a “no-drip, no-leak plug.”

The disclosure further provides numerous medical and veterinary uses for the compositions that benefit from the characteristics of the formulations. While human applications will become apparent from the disclosure, a preferred use relates to a unique method of forming a physical barrier in or on the teat (or a portion thereof) of a dairy animal for the prophylactic treatment of mammary disorders. The compositions can be particularly useful in the treatment of mammary disorders that typically occur as an animal begins to “dry-off” or during the time commonly referred to as the “dry period.” In some embodiments, methods of treatment consistent with the preferred uses of the compositions can comprise administering a composition as described herein to all or a portion of a teat, a teat canal, or a teat cistern of an animal. The composition is at a pH prior to injection such that the composition is a flowable liquid. A “flowable liquid” particularly will have a tan δ that is less than 0. After injection, the composition can be at a pH so that it rapidly transitions into a solid or semi-solid gel and thus can be configured to form a durable seal and/or a strong solid. A “solid gel” particularly will have a tan δ that is greater than 0. A “semi-solid gel” particularly will have a tan δ that is equal to 0. The presently disclosed compositions and methods can be particularly beneficial for reducing and/or preventing the invasion of the mammary gland by mastitis-causing microorganisms. Thus, the compositions and methods can be useful for reducing and/or preventing the incidence of new infections or reinfection in a non-human mammal.

This disclosure includes systems for forming a physical barrier, which is preferably an internal barrier within the teat canal, of a dairy animal to prevent mammary disorders or to lessen the harmful effects of infection. The system can comprise a composition as described herein and a delivery device for infusing the composition into the teat cistern of the animal. Such systems permit the treatment to block the invasion of the mammary gland by a mastitis-causing microorganism or to decrease the risk of the occurrence or re-occurrence of infection. More particularly, the present disclosure provides methods and systems wherein the composition is infused predominantly as the animal begins to dry off or during the dry period of a dairy livestock animal, preferably a heifer or a cow, but also can include other animals such as goats, sheep, water buffaloes, and the like. The composition can be configured to function as an aid in the prevention and the control of mastitis during the dry off period, thus reducing the clinical and sub-clinical cases during the dry off period and in the first stage (post calving) of lactation. By remaining in the teat canal throughout the dry period, the presently disclosed composition eliminates and/or reduces microbial invasion through the teat canal during high risk periods in the pre-fresh dairy animal.

In one or more embodiments, the disclosure can provide methods for combatting microbial mammary mastitis in a dairy animal which methods permit milk obtained from the animal to be used in the production of a milk product. Such methods can comprise applying topically or infusing a composition as described herein directly on the relevant mammary tissue of a dairy animal or within the teat canal to form a teat seal. The presently disclosed compositions thus effectively function as a teat sealant that can be provided via intramammary administration to each teat at the time of drying-off. Preferably, the present composition is applied or infused prior to infection of a healthy animal.

In further embodiments, the disclosure provides methods for reducing the withholding time of milk obtained from an animal being treated for mastitis before public consumption is allowed in the production of a milk product. Specifically, the presently disclosed composition can be applied topically to or infused within the teat canal of the animal.

In some embodiments, the present disclosure also provides methods for reducing the withholding time of milk obtained from an animal being prophylactically treated to prevent or to reduce the frequency of mastitis in order to improve the production of a milk product. Specifically, a presently disclosed composition can be applied topically to or infused within the teat canal of the animal.

The presently disclosed composition can be administered via intramammary infusion or by dipping the teat or other application method (e.g., wiping, spraying, or the like). Administration can be carried out as the animal begins to dry off or during the dry period. In some embodiments, however, the composition can be administered during the postpartum period of a non-lactating animal or during the prepartum period of an animal.

The presently disclosed compositions are beneficially capable of being prepared in the form of a flowable liquid that can easily be applied or otherwise administered to the appropriate tissue. The compositions, however, are further configured to quickly transition to a solid or semi-solid gel under the pH conditions of the treatment site. In particular, it is understood that the teat of a non-human mammal typically exhibits a pH in the range of about 6.8 to about 7.7. The present composition thus can be provided in a pH that is less than this range and be in the form of a flowable liquid in such lesser pH range. The composition is likewise capable of increasing in pH because of the nature of the materials utilized in the composition and/or the presence of pH-increasing moieties at the site of administration.

It should be appreciated that all scientific and technological terms used herein have the same meaning as commonly understood by those of ordinary skill in the art. The following definitions are given merely to illustrate the general meanings of the main terms used in connection with the present disclosure.

The term “udder” refers herein to the glandular, mammary structure of a female ruminant animal such as a cow, a goat, a sheep, a water buffalo, and the like. In the cow, the udder comprises four independent glands, with one teat and one exit duct each, whereas sheep and goat have two glands.

The term “teat” refers herein to the projecting part of the mammary gland containing part of the milk or teat sinus.

The term “teat sealant” refers herein to compositions and devices used to form a physical barrier on the surface of or inside an animal teat. A teat sealant can be on the teat surface, inside the teat streak canal, and/or inside the teat cistern.

The term “antimicrobial” refers herein to a substance that kills or inhibits the growth or reproduction of microorganisms such as bacteria, viruses, fungi, yeast, or protozoans.

The term “solution” refers herein to solutions, suspensions, or dispersions, unless otherwise stated.

The term “spray” as used herein refers to an atomized composition, such as comprised of small or large liquid droplets, such as applied through an aerosol applicator or pump spray applicator for the intended purpose of delivering a broad application of the composition.

The term “stream” refers herein to a continuous, direct, and focused application of the composition.

The term “infusion” refers herein to the continuous introduction of a fluid or solution into a cavity, vein or cistern.

The term “mammal” refers herein to a warm-blooded vertebrate animal of the class Mammalia, which includes both human and animal, that possess hair or fur on the skin, the secretion of milk from milk-producing mammary glands by females for nourishing the young, and a four-chambered heart.

For the embodiments of the disclosure that relate to mastitis, the term “animal” refers herein to a female, non-human mammal which has a lactation period, which includes, but is not limited to, livestock animals, such as cows, sheep, goats, horses, pigs, water buffaloes and the like. Preferably, the animal is a dairy cow. While both the “cow” and the “heifer” are female bovines, the term “heifer” refers herein to any young female cow that has not given birth to a calf, typically one that has been weaned and under the age of 3 years. The term “cow” often refers to an older female animal that has given birth to a calf.

The term “dry period” refers herein to the non-lactating phase of the lactation cycle of a cow or other dairy animal. The dry period occurs between the end of one lactation cycle and the beginning of the next lactation. At the end of each lactation cycle, the animal begins the phase of “drying off” as the animal enters the dry period which includes the usual physiological, metabolic and endocrine changes associated with cessation of milk production for the non-lactating period (dry period) of the animal.

The term “milk product” refers herein to a product containing any amount of milk in liquid or powder form. It also includes cheese and yogurt.

The term “postpartum” refers herein to the period of time beginning immediately after calving and extending for about six weeks.

The term “prepartum” refers herein to the period of time during pregnancy, which is prior to calving.

The term “periparturient” refers herein to the period immediately before and after calving.

The term “involution” refers herein to the first two to three weeks after cessation of milk production in a cow.

The term “keratin plug” refers herein to keratin-based occlusion of the teat canal/streak canal of a cow following cessation of milk production for the dry period.

The term “microbial invasion” refers herein to movement of pathogenic microorganisms such as, for example, bacteria, especially pus-forming or necrotizing bacteria, viruses, fungi, yeast, protozoans and the like that proliferate into bodily tissue or bodily cavities, resulting in tissue injury that can progress to infection and/or disease. For purposes of the disclosure, the “microbial invasion” typically refers herein to a “bacterial invasion.”

The term “solid” refers to a physical form that is non-flowable under the ambient conditions.

The term “semi-solid” refers to a physical form that is not solid but is sufficiently viscous to resist flowing under the ambient conditions.

The term “gel” refers to a substantially dilute, cross-linked system, which exhibits no flow when in the steady-state. By weight, the gel is mostly liquid yet behaves like a solid due to a three-dimensional cross-linked network within the liquid formed by a polymer present in the gel.

The terms “solidification” and “gelation” are used interchangeably herein to refer to the phase transition between the liquid state and the solid (or semi-solid) state.

The term “polymer” refers herein to a material that includes a set of macromolecules. Macromolecules included in a polymer can be the same or can be differ from one another in some fashion.

As used herein with reference to a polymer, the term “molecular weight (MW)” refers to a number average molecular weight, a weight average molecular weight, or a melt index of the polymer.

The term “elastic modulus” (also referred to as “Young's modulus” or the storage modulus (G′)) is defined herein as the change in stress with an applied strain (that is, the ratio of shear stress (force per unit area) to the shear strain (proportional deformation)) in a material. Essentially, the elastic modulus is a quantitative measurement of stiffness of an elastic material that measures the ability of the tested material to return to its original shape and size. G′ can be calculated using a formula derived from Hooke's law, which states that the elastic modulus is equal to the ratio of stress to strain (i.e., the ratio of applied pressure to fractional change in size). The measure of the elastic modulus is reported as the force per unit area (the standard metric ratio of the Newton to unit area (N/m²) or the pascal (Pa) in which one pascal is equivalent to one Newton (1 N) of force applied over an area of one meter squared (1 m²)). This pascal unit is an art-recognized term often used to define a unit of pressure, tensile strength, stress and elasticity.

The term “loss tangent tan δ” or “tan δ” refers herein to the tangent of the phase angle, that is, the ratio of viscous modulus (G″) to elastic modulus (G) and a helpful quantifier of the presence and the degree of elasticity in a fluid.

As used herein. “strong” is intended to mean the elastic modulus G′ that can generally range widely from about 420 Pa or higher, about 600 Pa to about 10,000 Pa, or about 6000 Pa to about 10,000 Pa. etc. at physiological temperature. Based on the level of stiffness, a solid body, for example, deforms when a load is applied to it. If the material is elastic, the body returns to its original shape after the load is removed. A “strong solid” is generally a solid or semi-solid gel for which G′ at physiological conditions (e.g., 37° C., and/or near physiological pH) is typically above about 560 Pa, although strong solids may form below about 560 Pa or above about 10,000 Pa depending on other factors in the processing steps to make, to sterilize or to store the formulation. The term “physiological temperature” used herein is intended to mean the normal body temperature range for the mammal.

The presently disclosed compositions include chitosan, which is a commercially available, relatively inexpensive polymer obtained by partial to substantial alkaline N-deacetylation of chitin, a linear polysaccharide, made of N-acetyl glucosamine units, linked via P-1,4-glycosidic bonds. The deacetylation process is generally performed using hot, concentrated, hydroxide solutions, usually sodium hydroxide. Chitosan is biocompatible, non-toxic, and non-immunogenic, allowing its use in the medical, pharmaceutical, cosmetic and tissue construction fields. Moreover, chitosan is metabolized-cleaved by certain specific enzymes, e.g., lysozyme, and can therefore be considered as biodegradable. In addition, chitosan is known to promote wound healing and exhibit antibacterial, antifungal, and antitumor properties.

For use in the present compositions, it can be preferred for the chitosan to exhibit at least a specified degree of deacetylation. In some embodiments, the chitosan has a degree of deacetylation (% DDA) of at least about 75%, at least about 77%, at least about 80%, or at least about 90% (e.g., with a maximum of 99.9% deacetylation). In some embodiments, the chitosan can have a % DDA of about 75% to about 99.9%, about 90% to about 99.9%, or about 95% to about 99.9%.

The chitosan is preferably present in an amount of about 1% by weight to about 10% by weight. In some embodiments, in can be preferred to include the chitosan in a relatively low concentration, such as about 1.5% to about 6%, about 2% to about 5%, or about 3% to about 5% by weight, based on the total weight of the composition.

In one or more embodiments, the present compositions may include one or more hydrophilic polymers in addition to chitosan. Examples of suitable hydrophilic polymers include, but are not limited to, methyl cellulose (MC) such as methyl cellulose ethers or cellulose ethers, polyvinyl acetate (PVA), PVA-acylate, hydroxypropyl cellulose (HPC), ethyl hydroxyethyl cellulose (EHEC), hyaluronic acid (HA), a poloxamer (a nonionic triblock copolymer) such as Pluronic®, sodium alginate, or another water-soluble polysaccharide capable of forming a thermosensitive gel exhibiting high viscosity at a sufficiently high pH. The hydrophilic polymer may be acylated. In some embodiments, it can be desirable to limit the content of solids in the composition that may unduly increase solution viscosity at the formation pH range. As such, one or more hydrophilic polymers (with the exception of chitosan) may be expressly excluded from the present compositions. For example, cellulosic materials can significantly stiffen some compositions at the formation pH range and thus may be expressly excluded. In some embodiments, the composition may be substantially free of any cellulosic materials (e.g., including less than 0.1 wt % cellulosic material). When present in addition to chitosan, the hydrophilic polymer may be present in a concentration of about 0.1% to about 20% by weight, based on the total weight of the composition.

The present compositions further can include one or more fillers. The fillers can be particularly useful to increase the density of the composition and thus ensure correct placement within a teat, for example. The filler can be a solid material and can be in particulate (e.g., powder) form. Preferably, the filler is non-soluble in substantially pure water or exhibits only low solubility (e.g., solubility of less than 25 grams per liter, less than 10 grams per liter or less than 1 gram per liter) in substantially pure water at 25° C. A material exhibiting no solubility or low solubility as defined above in substantially pure water may thus be characterized as being a water insoluble, solid filler. The filler should be non-toxic to mammals, and non-irritating in manners of use as described herein (e.g., non-irritating to the teat of a dairy animal). In some embodiments, the filler may be completely free or substantially free of any metals. The filler particularly can be an inorganic filler, such as an inorganic salt. Calcium salts in particular may be used. Non-limiting examples of materials suitable for use as fillers in various embodiments of the present compositions include calcium phosphate, aluminum oxide, calcium carbonate, silicon dioxide, and combinations thereof.

A single filler or a combination of fillers can be included in the composition in a total amount of at least 1%, at least 2%, at least 5%, or at least 20% by weight (e.g., with a maximum of about 50% by weight). In some embodiments, one or more fillers are present in a total amount of about 1% to about 40%, about 5% to about 40%, about 10% to about 40%, about 10% to about 30%, or about 15% to about 25% by weight, based on the total weight of the composition.

The content of filler(s) and the specific filler(s) used in the composition can be chosen in order to achieve a desired density for the composition. Preferably, the composition in liquid form has a density of at least 1.05 g/cm³, at least 1.1 g/cm³, or at least 1.2 g/cm³. In some embodiments, the density in the liquid form is about 1.05 g/cm³ to about 5 g/cm³, about 1.1 g/cm³ to about 4 g/cm³, or about 1.15 g/cm³ to about 3 g/cm³.

The compositions further include one or more biocompatible acids. In some embodiments, the biocompatible acids particularly can include organic acids, such as acetic acid. The acid is preferably present in an amount sufficient to lower the pH of the composition below a point such that the composition is in a flowable, liquid form. In some embodiments, the acid can be present in an amount of about 0.2% to about 10%, about 0.5% to about 5%, or about 1% to about 3% by weight, based on the total weight of the composition.

In some embodiments, the chitosan and the acid may be provided in a defined ratio. For example, the chitosan and the biocompatible acid can be present in the composition in a chitosan to acid weight ratio of about 1:1 to about 4:1. Preferably, the ratio of chitosan to acid is about 1:1 to about 3:1, about 1:1 to about 2:1, or about 1:1 to about 1.5:1.

The compositions further include one or more fluidizing agents. A suitable fluidizing agent can be a material that is useful to reduce solution viscosity at the formation pH. The properties of chitosan making it particularly useful in methods as described herein can result in compositions with initial viscosities that cause difficulty in delivery of the composition, such as through a syringe. Accordingly, the addition of one or more fluidizing agents can be beneficial to lower solution viscosity to improve flow properties of the liquid composition, such as syringeability. The fluidizing agent preferably is hydrophilic in nature and may be a polymeric material or non-polymeric. Non-limiting examples of fluidizing agents suitable for use according to the present disclosure include polyethylene glycol (PEG) and glycerol. A single fluidizing agent or a combination of fluidizing agents may be present in the composition in a total content of about 0.1 wt % to about 30 wt %, about 0.5 wt % to about 25 wt %, about 1 wt % to about 20 wt %, or about 5 wt % to about 15 wt %.

The amount of fluidizing agent may relate to the syringeability of the liquid composition. Preferably, the liquid composition exhibits sufficient flowability so that the liquid can pass through a syringe, having a cannula with an internal diameter of between about 1.25-2.0 mm, with an applied force of less than 200N, less than 175N. or less than 150N, such as about 10N to about 200N, about 25N to about 180N, about 60N to about 160N, or about 80N to about 140N.

The present compositions further include at least a minimum content of an aqueous medium. Substantially pure, or deionized, water may be utilized; however, other water-based media may be used. In some embodiments, the composition comprises at least 50% by weight, at least 55%, or at least 60% by weight of an aqueous medium based upon the total weight of the composition (e.g., with a maximum of 80% by weight). In particular, the aqueous medium can comprise about 50% to about 80%, about 55% to about 75%, or about 60% to about 70% by weight of the composition based on the total weight of the composition.

In one or more embodiments, the composition may also optionally include one or more pharmaceutical agents, particularly antimicrobial agents having antibacterial, antiviral, anti-mycotic or anti-parasitic activity, and the like. The pharmaceutical agent or agents will become trapped in the composition upon its formation and be released from the composition immediately or over a period of time. Since the typical offending pathogen in mastitis is bacterium, the composition may desirably contain the antibacterial agent. There are a variety of antibacterial agents available for use in animals. These antibacterial agents include, but are not limited to, the following: macrolides, for example, tulathromycin (Draxxin®), tildipirosin (Zuprevo®), tilmicosin (Micotil®), tylosin phosphate (Tylan®), and gamithromycin (Zactran®); cephalosporins, for example, ceftiofur sodium (e.g., Naxcel® and Excenel®), ceftiofur hydrochloride (e.g., Excenel RTU®, Excenel RTU EZ®, Spectramast®), ceftiofur crystalline free acid (Excede®), cefovecin sodium (Convenia®), and cefpodoxime proxetil (Simplicef®); lincosaminide antibiotics, for example, lincomycin (Lincomix®), pirlimycin hydrochloride (Pirsue®), and clindamycin hydrochloride (Antirobe®); fluoroquinolones, for example, danofloxacin (Advocin®), enrofloxacin (Baytril®), and marbofloxacin (Zeniquin®); and tetracyclines, for example, chlortetracycline, oxytetracycline, and doxycycline. Other antibacterial agents include, but are not limited to, a penicillin derivative such as amoxicillin trihydrate alone or with clavulonic acid (Clavamox®), spectinomycin (Adspec®), potentiated sulfonamides including trimethoprim/sulfadiazine (Tucoprim®) and sulfadimethoxine/ormetoprim (Primor®); chloramphenicol and its derivatives such as thiamphenicol and fluorinated synthetic analogs of thiamphenicol such as florfenicol (for example, Nuflor® and Nuflor® Gold). An antimicrobial agent may be administered simultaneously or sequentially with the compositions of the present disclosure.

The present composition can be particularly useful in light of the ability to closely control the physical form of the composition based upon pH. Highly deacetylated chitosan is poorly soluble in aqueous solution at approximately neutral pH; however, solubilization increases with a reduction in pH. It is commonly recognized that chitosan can be solubilized in a solution of 0.1 M acetic acid. It has been found according to the present disclosure, however, that it is possible to provide a gellable solution in a flowable liquid form for maintaining the composition below a defined pH range, such as less than 6.5, less than 6.0, or less than 5.5. Preferably, a composition according to the present disclosure is prepared to have a solution pH that is within the above range and, preferably, in a range of about 3.5 to about 6.0, about 4 to about 5.5, or about 4.5 to about 5.0. As such, the composition is a flowable liquid at a pH in any of the foregoing ranges. Likewise, the composition can be configured to be a solid or semi-solid gel at a pH that is higher than the selected, foregoing range. In particular, the composition can be configured to be a solid or semi-solid gel at a pH of 6.5 or greater or 7.0 or greater (e.g., up to a maximum of about 10). In specific embodiments, the composition can be configured to be a solid or semi-solid gel at a pH of 6.5 to about 9, about 6.7 to about 8.5, or about 7 to about 8.

When the composition is provided in a pH range so as to be a flowable liquid, the chitosan chains are partially or fully solvated. When the pH range is increased, however, at least partial desolvation occurs due particularly to the use of the highly deacetylated chitosan. As desolvation occurs, the chitosan polymer chains coming out of solution cause a stiffening of the composition and crosslinking of the polymer chains leads for formation of a solid or semi-solid gel.

The pH-dependent nature of the present composition can provide for several desirable manners for controlling gel formation. In some embodiments, it is possible to control gel formation by controlling the content of the multivalent salt that is included in the composition.

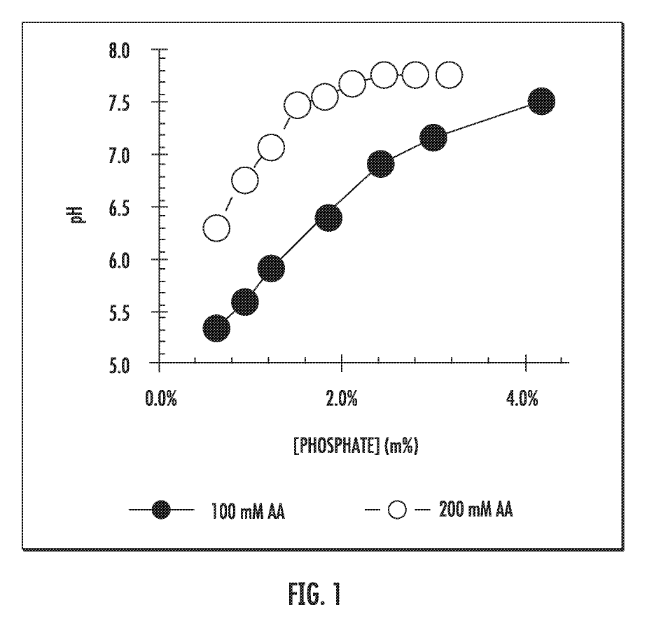

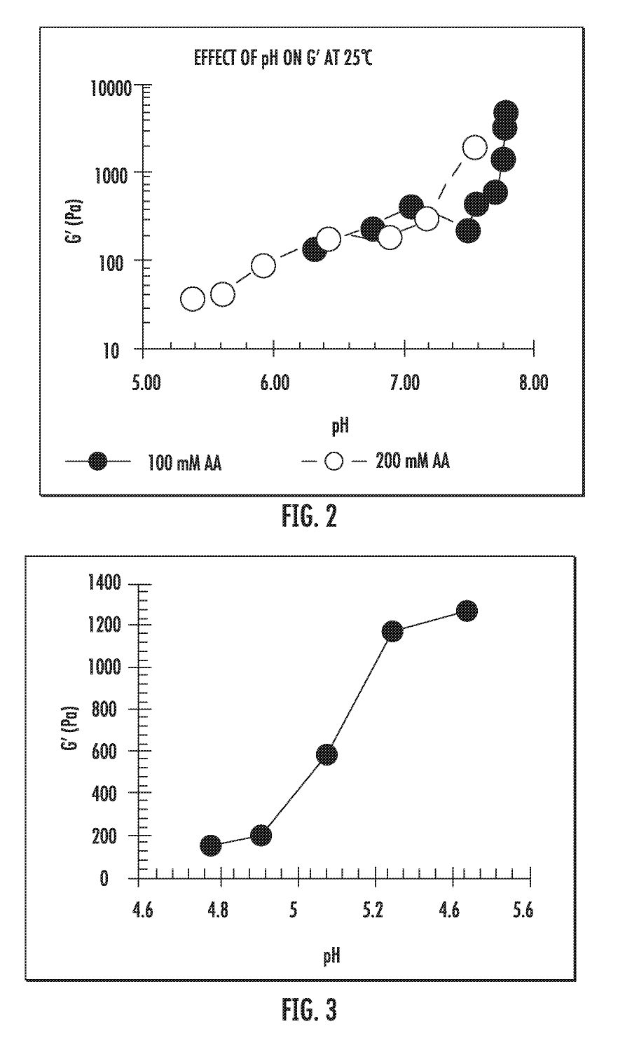

For example, FIG. 1 shows the correlation between dibasic phosphate salt content and pH for a composition comprising 44 g of water, 1 g of chitosan, 4 g methylcellulose, 100 or 200 mM acetic acid, and varying amounts of potassium phosphate dibasic salt. As seen in FIG. 1, an increase in the salt concentration generally correlated to a higher solution pH. As seen in FIG. 2, the higher solution pH also correlated to a stiffer gel. A rapid increase in stiffness was particularly seen at a pH of greater than about 7.

The effect of pH on gel stiffness is further evident in FIG. 3. Therein, compositions were prepared using 4 wt % chitosan, 2 wt % acetic acid, 20 wt % calcium phosphate dibasic, 2 wt % total monobasic and dibasic phosphate salts (K₂HPO₄/KH₂PO₄) and 72 wt % water. The ratio of the dibasic phosphate (pH 10.1) to monobasic (pH 4.1) anions (i.e., the K₂HPO₄/KH₂PO₄ ratio) was adjusted to achieve a constant 2 wt % phosphate content. As can be seen in FIG. 3, the gel stiffness significantly increased across a relatively narrow pH range of 4.78, 4.91, 5.08, 5.25, and 5.44. Based on the foregoing observations, testing as described in Example 1 was carried out to establish the ability to provide a pH-dependent gel suitable for use as a teat sealant.

In one or more embodiments, the compositions can be defined in relation to the qualitative analysis of the composition. Preferably, the gelled composition is configured to form durable seals. As such, it can be preferably for the gelled composition to exhibit an elastic modulus G′ (also referred to as the storage modulus (G′)) that is at least about 420 Pa or higher at physiological pH. Depending upon the animal treated, physiological temperature can range from about 37° C. to about 42° C. More specifically, G′ can be about 560 Pa or greater, about 600 Pa or greater, or about 450 Pa to about 10.000 Pa. Preferably. G′ for the gelled composition is about 500 Pa to about 5.000 Pa, or about 600 Pa to about 2,000 Pa. In some embodiments, G′ is about 450 Pa to about 600 Pa, about 500 Pa to about 1000 Pa, about 1000 Pa to about 6000 Pa, about 5000 Pa to about 9800 Pa, about 7000 Pa to about 10,000 Pa. or about 8500 Pa to about 10,000 Pa. In other embodiments. G′ for a composition that is particularly useful as a teat sealant may be about 420 Pa to 9,800 Pa.

Beneficially, the present compositions are configured for being infused directly into the teat canal of the milk-producing animal and forming a firm sealant during the dry period. The formulations can create this impermeable seal at physiological temperature in the presence of milk and under high ionic content that is usually seen upon the drying off of the mammary gland.

Depending on the delivery method, the composition in liquid form can be configured to have a desired viscosity to be made suitable for topical application, infusion and the like. Thus, the viscosity of the composition can be controlled so that the composition can be sprayed or streamed onto or into a teat in such a way that an excellent barrier is created. The liquid composition likewise may be passed through a syringe for quick and easy product use by the animal handler. In particular, the composition is beneficially capable of being infused using a single-barrel syringe. When infused directly into the teat, the formulations have the ability to fill the teat canal and transition to a solid or semi-solid gel at the increased pH level. Gelation of the composition in or on the teat can take place in a time of about 24 hours or less, such as about 1 hour to about 24 hours, about 4 hours to about 24 hours, or about 12 hours to about 24 hours.

Appropriate viscosity can depend upon the delivery means to be employed. In one or more embodiments, the composition can have a viscosity lower than about 800 cps at room temperature or during conditions of use (that is, not in the animal), preferably lower than 300 cps, more preferably lower than about 200 cps. Delivery through a pump spray normally requires a lower viscosity, such as less than about 150 cps. Spray without aerosol calls for a viscosity less than about 50 cps.

The present composition (e.g., in use as a teat sealant) can be applied to the teat by conventional means such as, for example, a spray or stream from a syringe, pump, spray nozzle, aerosol, dip, or other type of device. A combination of the spray and stream may be applied in a method similar to a shower head, whereby multiple streams provide the simulated broad coverage of a spray application. The composition can be sprayed or streamed externally onto the teat tissue whereupon it forms a barrier seal. For application by infusion inside the teat, as in the teat sinus or cistern, any veterinary syringe having a tapered syringe end, a teat needle, or an intramammary tip made especially for insertion of solutions in to the teat canal may be used. For an example, the liquid composition can be inserted through a conventional infusion cannula or infusion nozzle using a standard 5 or 6 mL syringe. An effective amount of the teat sealant that will form the desired physical barrier in a teat canal in order to prevent or treat a mammary disorder depends upon the dairy animal species and size of its teats. Typically, a volume of about 2 mL to about 3 mL is satisfactory to adequately fill the teat canal, but the amount may vary and can be easily titrated by the handler infusing the sealant into the teat. In some embodiments, about 0.5 to about 5.0 mL of the composition will be administered to an animal teat, preferably about 1.0 to about 4.0 mL, more preferably about 2.0 mL or higher, and even more preferably about 3.0 mL can be administered. Formulations may be pre-loaded into syringes for easy unit dose administration. Desirably, the composition is administered when the dairy animal enters into the dry period at the end of the lactation cycle or during the dry period of the animal, especially when it is a heifer or cow.

The composition may also be delivered externally or topically to the teat from a spray device or a stream device. The spray device may include a container having a dispenser for spray delivery of the liquid composition. The type of container used is variable, depending upon compatibility with the composition and the spray dispenser and can be glass, plastic, or metal. If the solutions are of a low enough viscosity, a spray delivery may be achieved with simple mechanical forces such as those achieved when depressing the plunger of a syringe by hand through an appropriately designed nozzle. It may be desirable to apply several layers of the composition to the teat to ensure adequate coverage of the teat. In any case, an effective amount for forming the physical barrier can be readily determined by visual appearance of the sealant on the teat.

The composition can also be delivered using a syringe outfitted with a spray head. Generally, any chemical, mechanical, or electronic method for propelling the liquid composition as a spray from the container is appropriate. In one aspect, a compatible liquid or gaseous aerosol propellant is placed in an appropriate container along with the composition and the dispenser includes a valve mechanism that enables atomized spray delivery of the liquid composition. Desirably, an intramammary infusion device may be used to deliver the teat sealant composition directly to the teat. The device can have a single dispenser, such as a spray tip from Nordson Corporation (Westlake, Ohio, U.S.A.). The device may include a meter so that the quantity of composition can be controlled.

Examples of devices that could be used, or modified for use, to deliver the compositions as teat sealants include those described in WO 2015/038281 (Zoetis). U.S. Patent Application No. 2015/0080841 (C. M. Bradley et al), U.S. Pat. No. 5,989,215 (Y. Delmotte et all), U.S. Pat. No. 8,353,877 (S. Hallahan et al), WO 2003/022245 (Bimeda Research & Development Limited), and WO 2013/021186 (Norbrook Laboratories Limited), the disclosures of which are incorporated herein by reference.

### Example

A composition was prepared using wt % chitosan, 20 wt % calcium phosphate (Ca₃(PO₄)₂), 64 wt % water, 2 wt % acetic acid, and 10 wt % PEG-400. The composition was stirred to form a uniform, flowable liquid solution with the properties shown below in Table 1.

TABLE 1
Physical Properties

| | |
|---|---|
| pH | 4.8-5.2 |
| Syringe Force | 80-140 N |
| Density | 1.24 (±0.09) g/mL |

Rheological Properties

| | |
|---|---|
| G′ at 25° C. | 205 Pa |
| G″ at 25° C. | 229 Pa |
| Tanδ at 25° C. | 1.12 |

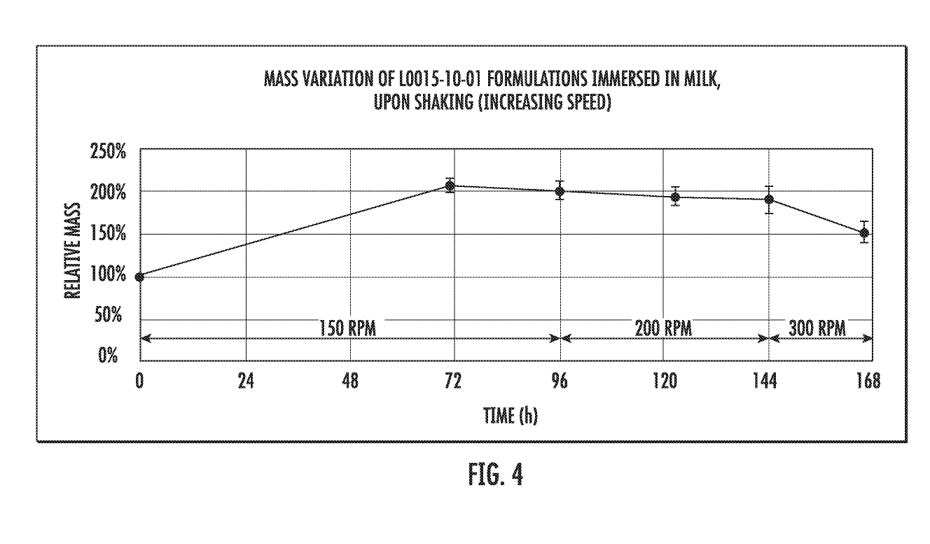

A semi-quantitative evaluation of the erosion and dissolution of the material in milk was carried out wherein gels formed with the above formulation were immersed in 37° C. milk and agitated for 7 days, during which the masses of the undissolved gels were periodically measured. The test results are shown in FIG. 4.

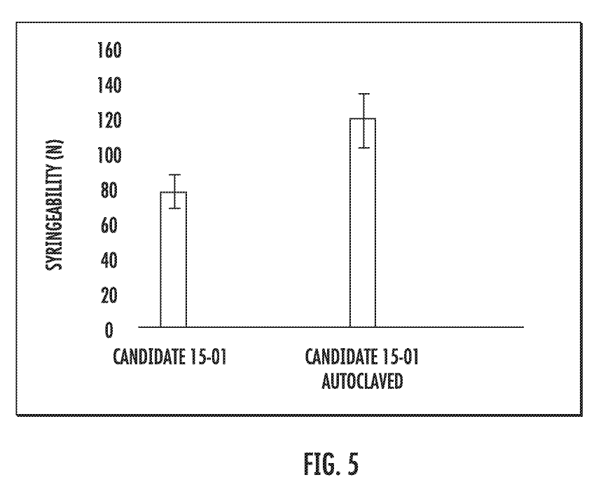

Syringeability of the formulation was evaluated to confirm ease of injection of the flowable liquid through a syringe. As seen in FIG. 5, syringeability was maintained within acceptable levels both before and after autoclaving.

1. A composition comprising:
chitosan;
a biocompatible acid;
a filler,
a fluidizing agent; and
at least 50% by weight of an aqueous medium based upon the total weight of the composition;
wherein the composition is a flowable liquid at a pH of less than 6 less and is a solid or semi-solid gel at a pH of about 6.5 or greater.

2. The composition of claim 1, wherein the chitosan is present in an amount of about 2% to about 5% by weight based on the total weight of the composition.

3. The composition of claim 1, wherein the chitosan has a degree of deacetylation of greater than 90%.

4. The composition of claim 1, wherein the biocompatible acid is present in an amount of about 0.5% to about 5% by weight based on the total weight of the composition.

5. The composition of claim 1, wherein the biocompatible acid is an organic acid.

6. The composition of claim 5, wherein the biocompatible acid is acetic acid.

7. The composition of claim 1, wherein the chitosan and the biocompatible acid are present in a chitosan to acid weight ratio of about 1:1 to about 4:1.

8. The composition of claim 1, wherein the filler is present in an amount of about 10% to about 40% by weight based on the total weight of the composition.

9. The composition of claim 1, wherein the filler is selected from the group consisting of calcium phosphate, aluminum oxide, calcium carbonate, silicon dioxide, and combinations thereof.

10. The composition of claim 1, wherein the fluidizing agent is selected from the group consisting of polyethylene glycol, glycerol, and combinations thereof.

11. The composition of claim 1, wherein the composition is substantially free of any cellulosic material.

12. A method of treating a non-human mammal, the method comprising administering a composition according to any of claim 1 through claim 11 to a teat, teat canal, or teat cistern of the non-human mammal.
